(19) United States
(12) Patent Application Publication
(10) Pub. No.: US 2015/0283022 A1
(43) Pub. Date: Oct. 8, 2015

(54) METALLIC HEATING/COOLING PRESSURE-REDUCTION SKIN MANAGEMENT DEVICE

(71) Applicant: UNISPATECH CO., LTD., Gangwon-do (KR)

(72) Inventors: Jung-Dong Lee, Wonju-si (KR); Sang-Sik Lee, Gangneung-si (KR)

(73) Assignee: UNISPATECH CO., LTD., Wonju-si (KR)

(21) Appl. No.: 14/431,919

(22) PCT Filed: Dec. 2, 2013
(86) PCT No.: PCT/KR2013/011058

§ 371 Date: Mar. 27, 2015

(30) Foreign Application Priority Data
Jan. 22, 2013 (KR) 10-2013-0007105

(51) Int. Cl. A61H 7/00; A61N 7/00

(52) U.S. Cl. CPC A61H 7/00; A61N 7/00; A61H 2201/0207; A61H 2201/0214

(57) ABSTRACT

Provided is a pressure-reduction skin management device, which includes: a suction unit for sucking the skin by applying a pressure reduction force to the skin so as to more effectively transfer a stimulus for skin management to the skin while sufficiently securing the pressure reduction force; and a skin stimulation unit provided on the suction unit, for applying energy to the expanded skin. The suction unit includes: a suction cup having a space formed therein and an open lower end to transfer the energy to the skin when coming into contact with the skin; and a housing surrounding the upperportion and the side surface of the suction cup while being spaced apart outward from the suction cup, the housing forming the outer shape of the suction unit. The skin stimulation unit includes a heating/cooling unit provided on the suction cup, for heating or cooling the suction cup.

### BACKGROUND OF THE INVENTION

1. Field of the Invention

The present invention relates to a device for the human body skin care. More specifically, the present invention relates to a metallic heating/cooling pressure-reduction skin management device to perform skin care on a skin by applying a reduced pressure.

2. Description of the Prior Art

There has been a growing importance of skin care recently as the interest increases in beauty care. The skin exists on the outer surface of the body and plays an important role of protecting internal organs from various external influences and maintaining life. Skin care, therefore, is more important than anything else.

Appropriate stimulation is required in addition to the skin cleanliness in order to prevent skin aging and maintain skin elasticity. Skin care devices have been developed and used to this aim by applying the wavelength of desired frequency, or heat and cold to the skin. In addition, devices with the ability to manage obesity are recently being developed by decomposing the subcutaneous tissue of the skin.

It is necessary, for a more efficient skin care, to apply a stimulus in a state of being inflated by suction of the skin through decompression.

Thus the present inventor developed a Massage device, which was filed in 2005 and registered in 2007 with Registration No. 0677863, which applied pressure and inflate the skin, and applied a stimulus on the expanded skin.

According to repetitive experiments, however, the conventional structure needs improvement because it has shortcomings including the difficulty to apply sufficient decompression, limited area of skin treatment, and a lowered efficiency due to poor transfer of the stimulus to the skin.

### TECHNICAL OBJECTIVES

Therefore, a metallic heating/cooling pressure-reduction skin management device shall be provided to more effectively deliver the skin care stimulus to the skin, while sufficiently securing the decompression.

Further, a metallic heating/cooling pressure-reduction skin management device is to be provided to increase the energy transfer efficiency to the skin.

### Problem-Solving Methodology

To this aim, the present skin management device comprises a suctioner for sucking the skin by applying the decompression to the skin, and comprises a skin stimulator, installed in the said suctioner, applying energy to the expanded skin; the said suctioner comprises a metal suction cup with an inner space and open bottom to contact with the skin and deliver energy, and a housing spaced apart from the said suction cup and forms the outer shape installed surrounding the top and side of the suction cup; and the said skin stimulator may comprise a hot/cold unit that is installed on the said suction cup and transfers cold or heat on the suction cup.

The said hot/cold unit may comprise a thermoelement installed on the outer surface of the said suction cup to transfer cold or warmth to the suction cup, and a water jacket installed on the outer surface of the said thermoelement to cool down the thermoelement.

The said hot/cold unit can be installed on two opposite sides of the said suction cup.

The said hot/cold unit may further comprise a vibration motor mounted on the said plate to vibrate the plate.

The said skin stimulator may further comprise a vibration element, coupled to the suction cup, for applying ultrasonic vibrations to the skin absorbed into the interior of the suction cup.

The said suction cup has an inner diameter gradually increasing from the top to the open bottom, has an air hole on the top for air flow, and can be integrated with a transfer plate on the outer surface in contact with the thermoelement to deliver heat or cold to the suction cup.

The suction cup may be made of a die cast aluminum material.

The said suction cup may have a structure that the open end is spread outward to form a skirt and a plurality of lugs are formed on the bottom of the said skirt.

The said suction cup may have a plurality of protruding lugs formed at intervals along the circumference of the said air hole in the top inner surface.

The said housing may comprise a neck formed at the top center and connected with the inside, and a handgrip formed on the said neck and extended to both sides around the neck.

The present device may further comprise a body connected to the said suctioner and skin stimulator for controlling the respective functions.

The said body comprises a case forming the outer shape, an air suction unit provided in the inner case and connected to the suction cup for applying suction pressure, a water cooler connected to the said water jacket for circulating the cooling water, a power supply electrically connected to the said thermoelement for applying current required for the thermoelement, a switch unit comprising switches for operating the respective driving units, a controller for controlling each component by computing operating signals of the said switch unit, and a display connected to said controller and comprises a monitor for displaying required data.

The said body may further comprise a power supply electrically connected to the said vibration element for applying the frequency and current needed for the vibration element.

The said air suction unit may comprise an air pump connected to the said suction cup through an air hose for selective intake of the air, and a solenoid valve provided on the said air hose for selective application of negative pressure and positive pressure to the suctioner through the air hose.

The present device may further comprise an energy unit capable of applying a quantum wave or low frequency wave, medium frequency wave, and a high-frequency wave including RF high frequency to the skin.

The said energy unit may comprise an oscillator to generate an oscillation voltage, and an output unit electrically connected to the said suction plate to supply the pulse output signal generating from the said oscillator.

### BRIEF DESCRIPTION OF THE DRAWINGS

FIG. 1 represents the perspective view of the metallic heating/cooling pressure-reduction skin management device in accordance with the present embodiment.

FIG. 2 represents the perspective view of the suction cup of the metallic heating/cooling pressure-reduction skin management device in accordance with the present embodiment.

FIG. 3 represents the bottom view of the suction cup of the metallic heating/cooling pressure-reduction skin management device in accordance with the present embodiment.

FIG. 4 represents the cross-section view of the metallic heating/cooling pressure-reduction skin management device in accordance with the present embodiment.

FIG. 5 represents the more specific cross-section view of the part of the suction cup of the metallic heating/cooling pressure-reduction skin management device in accordance with the present embodiment.

FIG. 6 represents the cross-section view of the suction cup of the metallic heating/cooling pressure-reduction skin management device in accordance with another embodiment.

FIG. 7 represents the schematic view of the connection structure of the metallic heating/cooling pressure-reduction skin management device and the body in accordance with the present embodiment.

### DETAILED DESCRIPTION

The embodiment of the present invention will now be described in more detail with reference to the various figures of the drawings so that those with ordinary skills in the art that the present invention belongs to can carry out with ease. The present invention may, however, be embodied in many different forms, without being limited to the embodiments set forth herein.

It is advised that the drawings are schematic and not to the scale. The relative dimensions and proportion of the parts in the drawings have been shown exaggerated or reduced in size for the sake of clarity and convenience in the drawing, and the dimensions are just exemplary and not limiting. In addition, for the same structure, elements or components shown in more than one figure, the same reference numerals are used to denote the corresponding or similar features in other embodiments.

Although not otherwise defined, all terms, including technical terms and scientific terms herein have the same meanings that those with ordinary skill in the technical field to which the invention pertains generally understand. Commonly used and pre-defined terms are interpreted have the meaning consistent with related art documents and presently disclosed information, and not interpreted to have ideal or very official meaning unless otherwise defined.

An ideal embodiment of the present invention is specifically described with reference to the perspective view. As a result, various modifications of diagrams, for example, changes in the production method and/or specifications are expected. Embodiments are thus not limited to the specific forms of the illustrated region. Therefore, the area shown in the figure is just approximate originally, and these forms are not intended to illustrate the precise shape of the area and not intended to narrow the scope of the present invention.

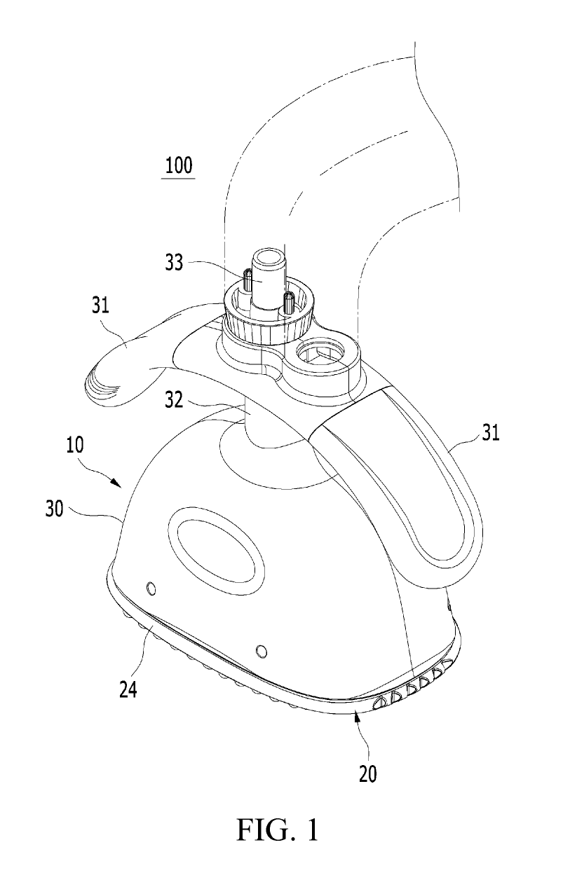

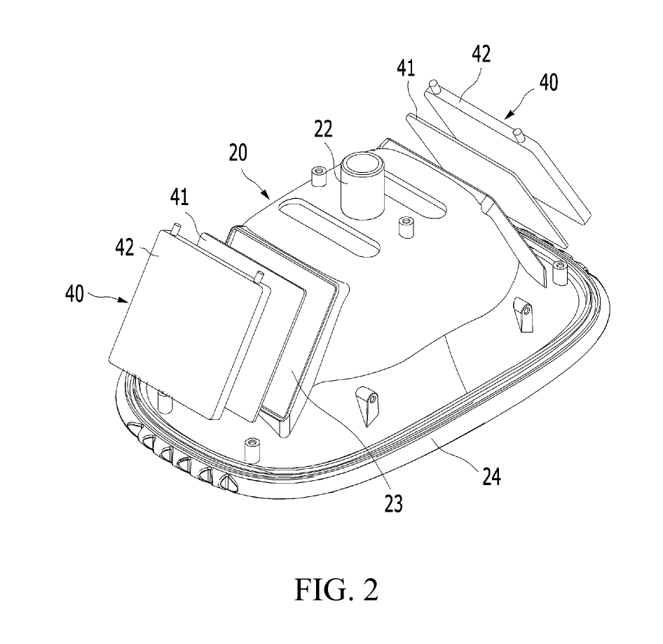

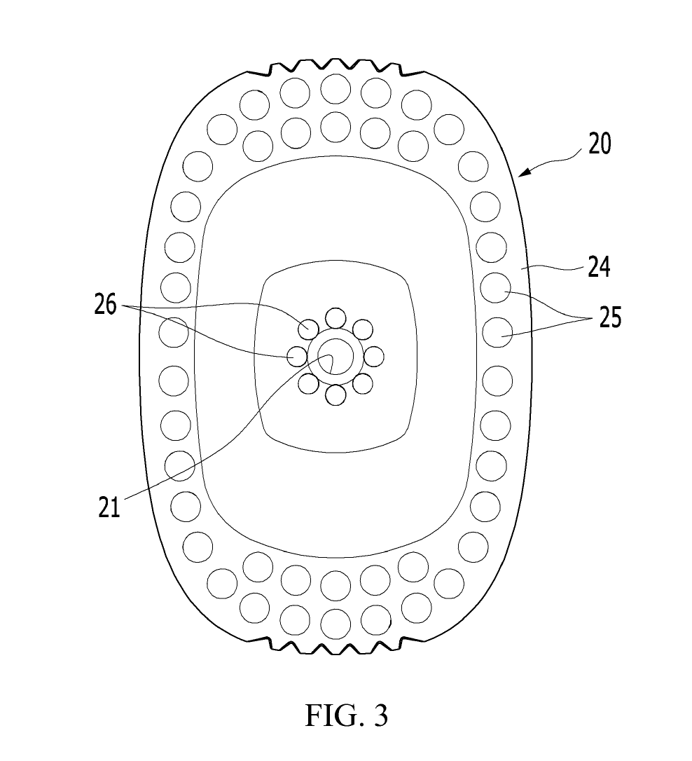

FIG. 1 shows the shape of the present embodiment metallic heating/cooling pressure-reduction skin management device in accordance with the present embodiment; FIG. 2 and FIG. 3 show the suction cup of the metallic heating/cooling pressure-reduction skin management device.

According to the above figure, the management device 100 comprises a suctioner 10 that sucks in the skin by applying decompression to the skin, and a skin stimulator installed in the said suctioner 10 for the heat or cold to the expanded skin.

In the description below, the decompression means to reduce the pressure to a negative pressure below atmospheric pressure.

The said suctioner 10 comprises a suction cup 20 and a housing 30 installed outside the suction cup.

The said suction cup 20 has an open lower end that is in contact with the skin and the shape of a container with a space inside. The skin is pushed and moved up to the inner space of the said suction cup 20. In the present embodiment, the said suction cup 20 has a rectangular shape with smoothly curved edge, and the shape of the said suction cup is not particularly limited. The said suction cup 20 has an inner diameter gradually increasing from the top to the open bottom, and has an air hole 21 on the top for air flow. The said suction cup 20 has a protruded connector 22, which is connected to the said air hole 21, on the outer top. The said suction cup 20 has an integrally provided transfer plate 23 on the outer surface, for installing a skin stimulator that applies cold or heat to the suction cup 20. In the present embodiment, the said transfer plate 23 constitutes a plane for the thermoelement (see 40 of FIG. 2) of the skin stimulator to be closely contacted.

The said transfer plate 23 is a position where the skin stimulator is installed, and can be formed on all the four sides and also on the top of the suction cup 20 in addition to the both sides.

The said suction cup 20 may be made of aluminum metal in the present embodiment. The suction cup 20 can be produced by aluminum die-casting. The suction cup 20 can be made of any die-castable metal with high thermal and electric conductivity, in addition to aluminum.

Like this, the suction cup, which contacts the skin 20, is made of metal to be able to play a role of an electrode that deliver energy such as heat or current, thus transfers energy evenly to the whole skin that is sucked into the suction cup 20.

As shown in FIG. 2 and FIG. 3, the open lower end of the said suction cup 20 is spread outwardly to form a skirt 24. In addition, a plurality of lugs 25 are formed at an interval on the lower surface of the said skirt 24 in contact with the skin. The said lug 25 has a structure protruding outwardly in the semi-spherical shape. The said lug 25 presses and stimulates the skin when the skin is sucked in.

In addition, as shown in FIG. 3, the said suction cup 20 may have the form with a plurality of lugs 26 projecting around the said air hole 21 at an interval in the upper inner side.

The said lugs 26 are formed projecting toward the bottom from the inside top of the suction cup 20. The said lugs 26 prevent the clogging of the air hole 21 by the skin sucked into the suction cup 20. When the skin is sucked into the suction cup 20 by the induced air through the air hole 21, the skin is gradually pushed up towards and near the air hole 21. At this time, the lugs 26 block the skin from coming close to the air hole 21 when the lugs 26 protruded around the air hole 21 contact the skin. Therefore, the skin is unable to proceed when in contact with the lugs 26. Because the said plurality of the lugs 26 are protruded from the suction cup 20 at intervals, gaps are formed between the lugs 26. Therefore, the air inside the suction cup 20 can continue to be discharged because the air hole 21 is connected with the inside of the suction cup 20 through the said gaps even if the skin contacts the front ends of the lugs 26.

The said lugs 26 are installed around the air hole 21 at intervals, and the interval or the degree of protrusion of the lugs 26 are not particularly limited. The front end of the said lugs 26 makes a spherical form, enabling smooth contact with the skin upon contact as much as possible.

The said housing 30 is provided spaced apart on the outside of the suction cup 20 and is installed wrapping around the top and side of the suction cup 20. The housing 30 is structured with its bottom open, and the skirt 24 at the bottom of the said suction cup 20 is exposed to the outside through the open lower end of the housing 30. The said housing 30 is made by injection molding of plastic, for example.

The said housing 30 is wrapping and protecting the suction cup 20 and the skin stimulator that is coupled to suction cup 20, forming the outer shape of the device. The handgrip 31 is integrally formed on one side of the housing 30 for easier to use of the present device. To this end, the neck 32, connecting to the inside, is formed extending upwards at the top center of the said housing 30; the handgrip 31, extending to both sides around the neck 32, is formed on the side of the said neck 32.

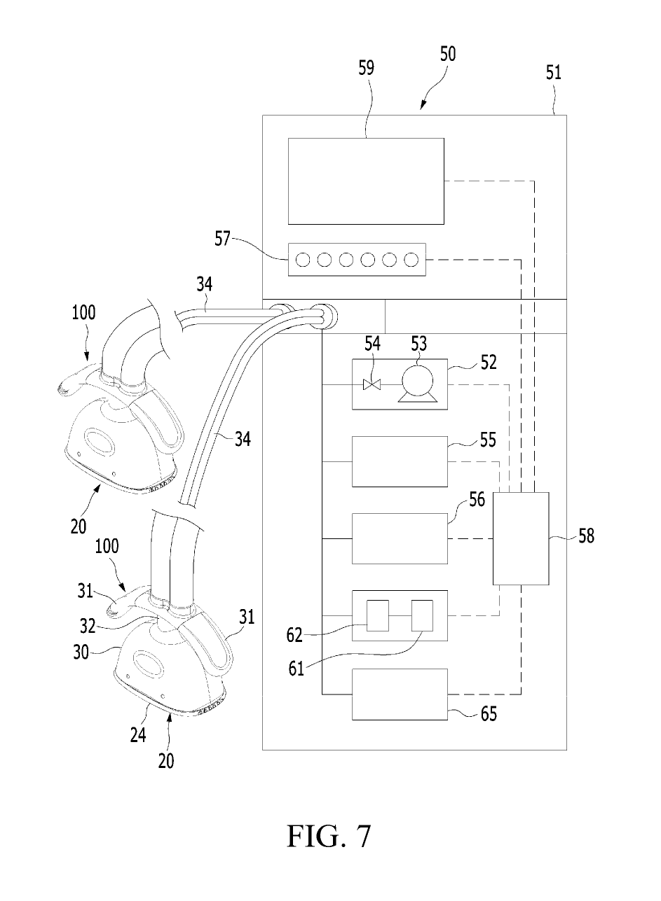

An air tube 33 is provided on the top of the said neck 32, for air distribution. In addition, the skin stimulator inside the housing is connected with the body (see 50 of FIG. 7) through a cable (see 34 of FIG. 7) of flexible material led from the said neck 32. The connection structure with the body will be described later.

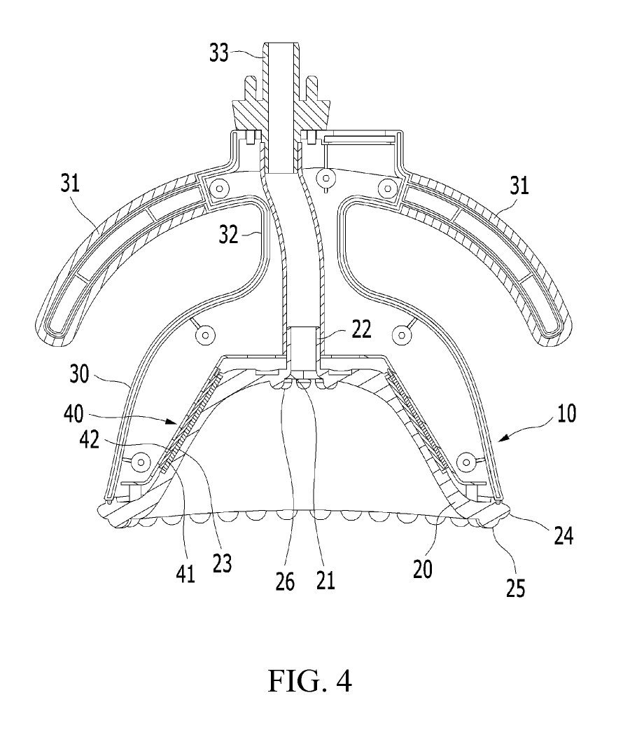

The decompression structure through the said suction cup 20 is as follows. As shown in FIG. 4, the said suction cup 20 is connected with the air tube 33 where the connector 22 formed in the top center air hole 21 is installed on the neck of the housing 32 by an air hose (not shown).

The decompression occurs when the air in the internal space of the suction cup 20 is discharged to the outside through the air tube 33 connected to the said air hole 21. The decompression is thus generated due to the negative pressure in the interior space of the suction cup 20, making the skin sucked in. Conversely, the internal pressure of the suction cup 20 increases by the flowing of the air into the suction cup 20 through the air hole 21 when air is supplied to the air tube 33. The suction force of the skin is therefore is reduced due to the decreased decompression of the suction cup 20.

The said skin stimulator comprises the hot/cold unit 40, installed on the said suction cup 20, to apply cold or heat to the suction cup 20.

The said hot/cold unit 40 comprises the thermoelement 41 installed on the transfer plate 23 of the said suction cup 20 to provide cold or heat to the suction cup 20, and the water jacket 42 installed on the outer surface of the said thermoelement 41 to cool down the thermoelement 41.

In the present embodiment, the said hot/cold units 40 are provided on both sides of the suction cup 20. The installation location of the said hot/cold unit is not particularly limited. The said hot/cold unit 40 may be installed on the top as well as on the four sides of the suction cup 20.

The said thermoelement 41 is installed in close contact with the transfer plate 23 formed integrally with the suction cup 20.

Because the said thermoelement 41 is a device using the cooling effect produced when the bipolar-type semiconductor is combined, that is, potential energy of electrons in the metal differs in different metals, it is necessary to obtain the external energy for carrying the electrons from the metal with the lower potential energy to the metal with the higher potential energy, so the thermal energy is lost at the interface or the heat energy is released vice versa, and the rise and fall of the temperature is done by switching the direction of current in accordance with this principle. Description will be omitted since many techniques are already disclosed of the said thermoelement 41 at the level of ordinary skill in the art.

Like this, cold and heat can be applied to the skin through the front face of suction cup 20 where the transfer plate 23 is formed because the said thermoelement 41 in itself generates heat in the heat generating surface and cold in the cold generating surface depending on the direction of the current.

In other words, the heat or cold of the thermoelement 41 transferred to the transfer plate 23 is passed to the front face of the suction cup 20 integral with the transfer plate 23 because the said suction cup 20 is made of a metal integrally formed with the transfer plate 23. Thus, the whole suction cup 20 is cooled or heated at a uniform temperature. Therefore, heat or cold can be applied to the whole skin that is sucked up into the inside of the suction cup 20 and contacting the inner surface of the suction cup 20.

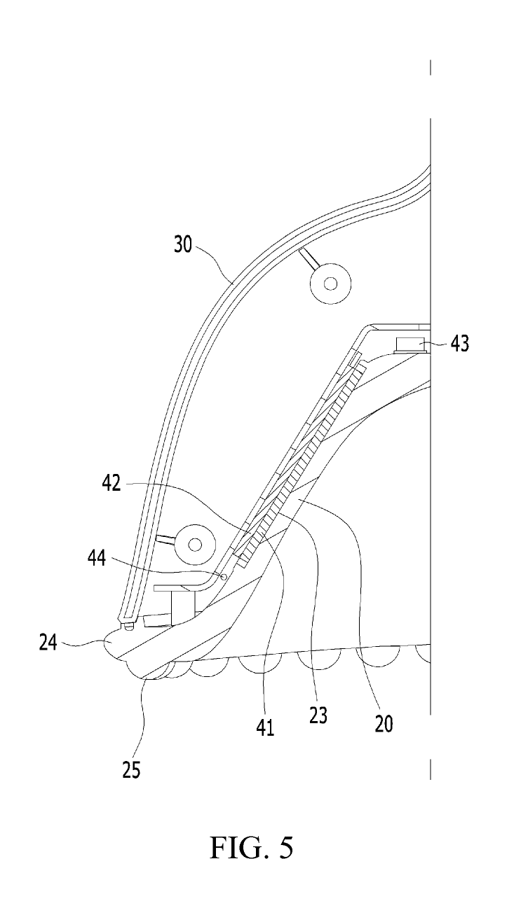

Here, the said hot/cold unit 40 has a structure to vibrate the skin through the transfer plate (23) when necessary, as well as applying the cold and heat to the skin. To this end, as illustrated in FIG. 5, the said hot/cold unit 40 is provided outside the said suction cup 20 and further comprises a vibration motor 43 for vibrating the suction cup 20.

The said vibration motor 43 has, for example, a structure to vibrate the object through the vibration generated by the vibrator during the motor drive because a vibrator is installed to the rotation shaft of the DC motor. In the present embodiment, the installation location, size or capacity of the said vibration motor 43 is not particularly limited if it is able to vibrate the suction cup 20. The wire which is connected to the said vibration motor 43 is connected to the body through the neck 32 of the housing 30, so power can be supplied from the body 50. Thus the suction cup 20 vibrates when the said vibration motor 43 is driven, and exerts a vibration energy to the skin that is sucked into and contacts the suction cup 20. Accordingly, the present device applies heat or cold to the skin in a state of vibration through the suction cup 20, resulting in the increased skin stimulating effects by the heat and cold can further.

In addition, a temperature sensor 44 is installed on one side of the said suction cup 20, so it is possible to measure the temperature of the suction cup applying cold or heat to the skin. The installation position of the said temperature sensor 44 is not particularly limited.

A water jacket 42 in the installed to the thermoelement 41 in contact with the exterior side of the said transfer plate 23, so the heat generated outer surface of the thermoelement 41 is radiated in the course of cooling or heating of the suction cup 20.

A passage is formed inside the said water jacket 42 for the circulation of the cooling water, and a supply port and a discharge port are provided outside for the supply and discharge of the cooling water.

The water tube connected to the supply port and discharge port of the said water jacket 42 is extended through the neck 32 of the housing 30 and connected to the body 50. In addition, the wire connected to the said thermoelement 41 and the temperature sensor 44 is also extended through the neck 32 of the said housing 30 and is connected to the body 50.

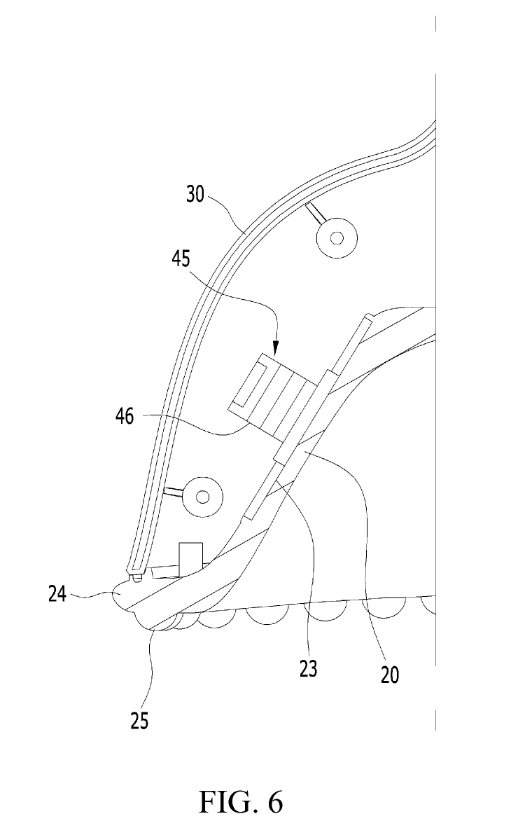

FIG. 6 shows another embodiment of the skin stimulator of the decompression skin management device.

Hereinafter, other components, other than the skin stimulator, of the present embodiment have the same structure that was described above. Therefore, same numerals are used for the same structure, of which the detailed description is omitted.

As shown in FIG. 6, the metallic heating/cooling pressure-reduction skin management device 100 of the present embodiment comprises the skin stimulator that is installed in the suction cup 20 to apply energy to the expanded skin; the said skin stimulator has the structure to contain a vibration unit 45 for applying ultrasonic vibrations to the skin that is absorbed into the interior of the said suction cup 20. The said vibration unit 45 comprises a vibration element 46, installed on the outer surface of the transfer plate 23 of the suction cup, for applying ultrasonic vibration to the skin through the said suction cup 20. The said vibration element is mounted on the outer surface of the transfer plate 23 of the said suction cup 20. The said vibration element 46 exerts skin stimulation by ultrasonic vibration to the skin in contact with the inner surface of the suction cup 20.

The said vibration element 46 may have the structure for applying the ultrasonic waves with a frequency range of 27˜40 KHz to the skin. Whereby it generates the cavitation effect peculiar to the ultrasonic waves of the frequency.

The metallic suction cup 20 plays a role of an electrode itself, being able to pass the ultrasonic vibration to the skin. Thus, the ultrasonic vibration generated from the vibration element 46 is transmitted to the suction cup 20 through the transfer plate 23 and applied to the skin over the entire inner surface of the suction cup 20.

The wire connected to the said vibration element 42 extends through the neck 32 of the said housing, and is connected to the body 50.

In the present embodiment, the said vibration element 46 may be installed on both the opposite sides of the suction cup 20. And the said hot/cold unit 40 may be installed on top of the suction cup 20. In addition to such structure, the vibration unit 45 may be provided only on one side of the suction cup 20, and the hot/cold unit 40 can be installed on the other side. Thus, by having the vibration unit and the hot/cold unit at the same time, the device can exert ultrasonic vibration to the skin by the vibration unit 45 through the suction cup 20, as well as the heat and cold by the hot/cold unit 40.

On the other hand, FIG. 7 shows the state of the metallic heating/cooling pressure-reduction skin management device, connected with the body 50, in accordance with the present embodiment.

As shown in the figure, the cable 34 is installed on the top of the neck 32 of the said housing 30, being connected to the body 50. The said cable has a flexible structure; for example air hose, water pipe and various wires required for the management device are extended inside.

The said body 50, which drives and controls all components of the present management device including the said suctioner 10, a hot/cold unit 40, and a vibration unit, contains a case 51 forming the appearance, an air suction unit 52 to suck in air through the said air hose provided in the said case 51, a water cooler 55 installed inside the said case 51 to feed and circulate cooling water to a water-cooling jacket 42 of the said hot/cold unit 40 through the said water pipe, a power supply 56 electrically connected with the said thermoelement 41 for applying electric current required for the thermoelement 41, a switch unit for operating each drive unit 57, a controller 58 for controlling each component by computing the operation signal of the said switch unit 57, and a display 59 connected to the said controller 58 for displaying the required data.

In addition, the body further comprises a power supply 65 electrically connected to the said vibration element 42 inside applying necessary current to the vibration element 42 to drive the said vibration unit 41. It is controlled in connection to the said power supply 65 controller 58.

The said air suction unit 52 may comprise an air pump 53 connected to the said suction cup 20 through an air hose for selective air intake, and a solenoid valve 54 installed on the said air hose for alternately applying positive and negative pressure to a suctioner 10 by the air hose.

Thus, the air suction unit 52 repeatedly mitigates the skin suction by iteratively applying negative and positive pressure to a suction cup 20 through the driving of the solenoid valve 54, enabling to prevent skin damage due to the decompression and more effective skin care thanks to the skin vibration.

Here, the present management device may have a structure to apply either one of the quantum wave or low frequency wave, medium frequency wave, or high-frequency wave including RF high frequency wave to the skin through the said suction cup 20.

To this end, the said suction cup 20 is connected to the body 50 with a wire for the current application. In addition, the said body 50 is equipped with an oscillator 61 generating the oscillation voltage to apply quantum wave or low frequency wave, medium frequency wave, or high frequency wave including RF high frequency wave to the suction cup 20, and an output unit 62 supplying the pulse output signal generating from the oscillator to the suction cup 20. The wire connected to the said suction cup 20 extends through the cable and is connected to the output unit 62 provided in the body 50.

The present management device has a structure to apply, for example, RF high frequency wave to the skin through a suction cup 20. Thus, when the user operates the switch on the body 50, the said oscillator 61 generates the changed output pulse in accordance with the signal of the controller 58 and supplies it to the suction cup 20 via the output unit 62. Therefore, the said suction cup 20 is able to apply the wavelength of a predetermined frequency, for example, the RF high frequency wave to the skin absorbed and contacted with the inside of the suction cup 20.

Here, the present management device can apply a RF high frequency wave to the skin in monopolar mode or bipolar mode; the current can be applied to the skin through the suction cup, which is an electrode, in a CET (Capacity Electric Transfer) scheme, as well as a RET (Resistive Electric Transfer) scheme. For a bipolar mode, two suction cups are equipped and each suction cup is used as the bipolar electrode. For the RET scheme, a metal suction cup 20 without insulation coating is used. The energy can be transferred deep into the fat layer of the skin, by applying a RF high frequency wave to the skin in a RET scheme through the suction cup. The operation of the present device is described as follows.

The present device is able to stimulate the skin by applying cold or heat, or a wavelength with a predetermined frequency to the skin through the suction cup 20 itself in a state that the skin is sucked into the suction cup 20.

The air pump 53 is operated driven by the equipment of the body 50, and the air inside the suction cup 20 is sucked out via the air hose connected to the air pump 53 in accordance with the opening and closing operation of the solenoid valve 54.

Thus, the skin in close contact with the suction cup 20 is pushed up into the suction cup 20 by negative pressure applied in the suction cup 20. The skin is sucked up into suction cup 20 to be in a state of completely contact inside.

In this state, the thermoelement 41 of the said hot/cold unit 40 is operated to cool the suction cup 20 where the transfer plate 23 is formed, and the suction cup 20 stimulates the skin by cooling the skin that is in close contact.

In addition, if a vibration unit 45 is furnished, stimulus can be applied to the skin closely contacting the suction cup 20 by applying ultrasonic vibration to the suction cup 20 through the drive of the vibration unit 45 while applying cold or heat to the skin through the suction cup 20. When the ultrasonic vibration is applied to the skin through the suction cup 20 according to the drive of the said vibration unit 45, bubbles are generated by the cavitation phenomenon due to the cavitation effect in the fat cells of the inner skin. The air bubbles generated in the skin inject a powerful energy during the rupture. Thus, the said bubbles easily destroy fat cells while bursting around the cell membrane of the fat cells.

Thus, the present device is able to apply energy such as cold or heat evenly throughout the extruded skin sucked inside the suction cup 20 by using the entire suction cup 20 as the electrode for energy delivery. Therefore, the skin stimulating effect can be enhanced.

It has been described in the above with respect to preferred embodiments of the present invention, the present invention is not limited thereto, and is possible to carry out in various modifications within the scope of the Claims, Disclosure of Invention and accompanying drawings; it is natural that this also falls in the scope of the present invention.

Reference Numeral

| | |
|---|---|
| 10: suctioner | 20: suction cup |
| 21: air hole | 22: connector |
| 23: transfer plate | 24: skirt |
| 25, 26: lug | 30: housing |
| 31: handgrip | 32: neck |
| 33: air tube | 34: cable |
| 40: hot/cold unit | 41: thermoelement |
| 42: water jacket | 43: vibration motor |
| 44: temp sensor | 45: vibration unit |
| 46: vibration element | 50: body |
| 51: case | 52: air suction unit |
| 53: air pump | 54: solenoid valve |
| 61: oscillator | 62: output unit |

### INDUSTRIAL APPLICABILITY

The device in this way increases the suction force of the skin, being able to raise the skin to the inner top of the suction cup to deliver energy to the skin more efficiently. Therefore, it is more effective for skin care owing to the more efficient and reliable stimulation to the skin. In addition, it can provide the best possible suction force regardless of the size, shape and state of the skin to care, enabling a uniform skin care.

In addition, it exerts the negative pressure and positive pressure repeatedly to the skin, preventing damage to the skin due to the decompression and enabling more effective skin care owing to the skin vibration.

In addition, the suction cup itself plays the role as an electrode for delivering energy to the skin, applying energy to the skin through the front face of the suction cup. Thus, the device can apply the energy to the skin in a uniform overall amount in a wider area, enhancing the massage effect.

What is claimed is:

1. A metallic heating/cooling pressure-reduction skin management device comprising:
a suctioner that absorbs the skin by applying the decompression, and a skin stimulator installed to the said suctioner for applying the energy to the expanded skin;
wherein the said suctioner comprises a metal suction cup with a space inside and open bottom for contacting the skin and delivering energy, and a housing spaced outwardly from the said suction cup and installed surrounding the top and side of the suction cup for forming the appearance; and
wherein the said skin stimulator comprises a hot/cold unit installed to the said suction cup, for applying a cold or heat to the suction cup.

2. The metallic heating/cooling pressure-reduction skin management device of claim 1, wherein
the said hot/cold unit comprises a thermoelement installed on the outer surface of the said suction cup for providing cold or heat to the suction cup, and a water jacket on the outer surface of the said thermoelement for cooling the thermoelement.

3. The metallic heating/cooling pressure-reduction skin management device of claim 2,
wherein the said hot/cold units are mounted on two opposite sides, facing each other, of the said suction cup.

4. The metallic heating/cooling pressure-reduction skin management device of claim 2,
wherein the said hot/cold unit comprises a vibration motor installed to the said suction cup for vibrating the suction cup.

5. The metallic heating/cooling pressure-reduction skin management device of claim 1,
wherein the said skin stimulator further comprises a vibration unit combined with the said suction cup for applying ultrasonic vibration to the skin absorbed inside the said suction cup.

6. The metallic heating/cooling pressure-reduction skin management device of claim 1,
wherein the said housing comprises a neck formed in the top center for communicating with the interior, and a handgrip formed in the said neck for extending to both sides around the neck.

7. The metallic heating/cooling pressure-reduction skin management device of either claim 1 or claim 6,
wherein the said suction cup whose inside diameter gets bigger from the top to the open bottom has an air hole formed at top for air circulation, and an integrated transfer plate contacting a thermoelement on the outer surface and conducting cold or heat to the suction cup.

8. The metallic heating/cooling pressure-reduction skin management device of claim 7,
wherein the said suction cup was made of die-cast aluminum. decompression skin.

9. The metallic heating/cooling pressure-reduction skin management device of 8,
wherein the said suction cup has structure that the open bottom spreads outward to form a skirt, and a plurality of lugs are formed at intervals at the bottom of the said skirt in contact with the skin.

10. The metallic heating/cooling pressure-reduction skin management device of claim 9,
wherein the said suction cup is structured that a plurality of projecting lugs are formed at intervals along the periphery of the said air hole on the inner top surface.

11. The metallic heating/cooling pressure-reduction skin management device of claim 10,
wherein a body, connected to the said suctioner and skin stimulator, is further contained for controlling the respective functions, and
wherein the said body comprises a case forming the outer shape, an air suction unit provided in the inner case and connected to the suction cup for applying suction pressure, a water cooler connected to the said water jacket for supplying and circulating the cooling water, a power supply electrically connected to the said thermoelement for applying current required for the thermoelement, a switch unit comprising switches for operating the respective driving units, a controller for controlling each component by computing operating signals of the said switch unit, and a display connected to said controller and comprises a monitor for displaying required data.

12. The metallic heating/cooling pressure-reduction skin management device of claim 11,
wherein the said body further comprises a power supply electrically connected to the said vibration element for applying current of required frequency for the vibration element.

13. The metallic heating/cooling pressure-reduction skin management device of claim 11,
wherein the said air suction unit comprises an air pump connected to the said suction cup through an air hose for selective intake of the air, and a solenoid valve provided on the said air hose for selective application of negative pressure and positive pressure to the suctioner through the air hose.

14. The metallic heating/cooling pressure-reduction skin management device of claim 11,
wherein the said skin stimulator further comprise an energy unit capable of applying a quantum wave or low frequency wave, medium frequency wave, or a high-frequency wave including RF high frequency to the skin,
wherein the said energy unit comprises an oscillator connected to the said body to generate an oscillation voltage, and an output unit electrically connected to the said suction to supply the pulse output signal generating from the said oscillator.
